(19) United States
(12) Patent Application Publication
(10) Pub. No.: US 2024/0341663 A1
(43) Pub. Date: Oct. 17, 2024

(54) CATHETERS WITH REINFORCED SHEATH HAVING A COUPLED TUBE

(71) Applicant: Boston Scientific Medical Device Limited, Galway (IE)

(72) Inventors: Miles Montgomery, Holland Landing (CA); Ros Thomas Brannick, Picton (CA); Luke Keaveney, Georgetown (CA); Kevin Banks, Ballinamore (IE); Melaine Dhaliwal, Brampton (CA); Gopi Shah, Brampton (CA); Suresh Subramaniam, Cambridge (CA)

(21) Appl. No.: 18/632,887

(22) Filed: Apr. 11, 2024

Related U.S. Application Data
(60) Provisional application No. 63/495,746, filed on Apr. 12, 2023.
(60) Provisional application No. 63/592,780, filed on Oct. 24, 2023.

(51) Int. Cl. A61B 5/367; A61B 5/00; A61B 5/339; A61B 34/20; A61B 90/00

(52) U.S. Cl. CPC A61B 5/367; A61B 5/339; A61B 5/6852; A61B 34/20; A61B 90/39; A61B 2034/2051; A61B 2090/3966; A61B 2560/0468; A61B 2562/043; A61B 2562/222; A61B 2562/227

(57) ABSTRACT

A catheter is disclosed. The catheter includes an elongated shaft defining a lumen and having a distal portion. The elongated shaft includes an outermost elongated support member coaxial with the lumen. The support member includes an outer side. An electrode is disposed on the distal portion of the elongated shaft. An elongated tube extends longitudinally along the elongated shaft, and the elongated tube interfaces with the support member along the outer side. A lead conductor is disposed within the tube and electrically coupled to the electrode. A coupling member is disposed about the tube and outer side of the support member such that the coupling member couples the tube to the support member.

### CROSS REFERENCE TO RELATED APPLICATIONS

This application claims the benefit and priority to U.S. Provisional Application 63/495,746 entitled “CATHETERS WITH REINFORCED SHEATH HAVING COUPLED TUBE,” filed Apr. 12, 2023, and U.S. Provisional Application 63/592,780 entitled “CATHETERS WITH REINFORCED SHEATH HAVING COUPLED TUBE,” filed Oct. 24, 2023, which are incorporated in their entireties herewith.

### TECHNICAL FIELD

The present disclosure relates to medical devices for catheterization procedures, such as medical devices for electrophysiological procedures. More specifically, the present disclosure relates to catheters and methods for manufacturing catheters.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths, dilators, and other medical tools, which can be referred to as catheters in this disclosure. Electrophysiological procedures can involve the visualization of the heart, heart activity, and the position of the catheters within the heart. A common visualization system involves the use of fluoroscopy, which can expose the patient and clinician to radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheters for positioning and manipulation. Catheters used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheters include magnetic sensors. Impedance-based catheters use electrodes in the presence of electric fields to track the catheters.

### SUMMARY

In an Example 1, a catheter comprising an elongated shaft defining a lumen and having a distal portion, the elongated shaft having an outermost elongated support member coaxial with the lumen, the support member having an outer side; an electrode disposed on the distal portion of the elongated shaft; an elongated tube extending longitudinally along the elongated shaft, the elongated tube coupled to the support member along the outer side; a lead conductor disposed within the tube and electrically coupled to the electrode; and a coupling member disposed about the tube and outer side of the support member, the coupling member coupling the tube to the support member.

In an Example 2, the catheter of Example 1, wherein the support member includes a braided material forming a braided member.

In an Example 3, the catheter of Example 1, wherein the coupling member includes one or more coils wound around the elongated tube and support member.

In an Example 4, the catheter of Examples 3, wherein the elongated tube transitions from under to over the coupling member on the support member.

In an Example 5, the catheter of any of Example 1-4, wherein the shaft includes a cover disposed on the coupling member, tube, and support member.

In an Example 6, the catheter of Example 5, wherein the cover includes a polyether block amide and the coupling member includes nylon.

In an Example 7, the catheter of any of Example 1-6, wherein the elongate tube includes a plurality of elongate tubes spaced-apart on the outer side.

In an Example 8, the catheter of any of Example 1-7, wherein the elongated tube interfacing with the braided member only along the outer side.

In an Example 9, the catheter of Example 1, wherein the electrode is ring electrode.

In an Example 10, the catheter of Example 1, wherein the elongated tube includes a mandrel disposed within the tube for enabling winding of the coupling member around the tube and the outer side of the support member.

In an Example 11, the catheter of any of Examples 1 or 10, wherein the tube includes a tube distal end, wherein the tube distal end is closed prior to a cover being applied.

In an Example 12, the catheter of Example 11, wherein the tube distal end is closed via the coupling member wound around the tube distal end and on outer side of the support member.

In an Example 13, the catheter of Examples 1, wherein the shaft includes one support member.

In an Example 14, the catheter of any of Examples 1 and 13, wherein the support member includes in inner surface, and the inner surface includes a liner layer.

In an Example 15, the catheter of any of Examples 1-14, wherein the catheter is a guide catheter having a proximal end coupled to a handle.

In an Example 16, a catheter comprising an elongated shaft defining a lumen and having a distal portion, the elongated shaft having an outermost elongated support member coaxial with the lumen, the support member having an outer side; an electrode disposed on the distal portion of the elongated shaft; an elongated tube extending longitudinally along the elongated shaft, the elongated tube coupled to the support member along the outer side; a lead conductor disposed within the tube and electrically coupled to the electrode; and a coupling member disposed about the tube and outer side of the support member, the coupling member coupling the tube to the support member.

In an Example 17, the catheter of Example 16, wherein the support member includes a braided material forming a braided member.

In an Example 18, the catheter of Example 17, wherein the braided member includes conductive fibers.

In an Example 19, the catheter of Example 18, wherein the coupling member is a thread.

In an Example 20, the catheter of Example 16, wherein the elongate tube traverses into the lumen through the braided member on a proximal side of the shaft.

In an Example 21, the catheter of Example 16, wherein the shaft includes a cover disposed on the coupling member, tube, and support member.

In an Example 22, the catheter of Example 21, wherein the cover includes a polyether block amide and the thread includes nylon.

In an Example 23, the catheter of Example 16, wherein the elongate tube includes a plurality of elongate tubes radially spaced-apart on the outer side.

In an Example 24, the catheter of Example 16, wherein the support member is a liner layer.

In an Example 25, the catheter of Example 16, wherein the coupling member includes one or more coils wound around the elongated tube and support member.

In an Example 26, the catheter of Example 25, wherein the coupling member includes a first coil and a second coil, the first coil wound in a first direction along a length of the support member and the coil wound in a second direction along the length of the support member, the second direction opposite the first direction.

In an Example 27, the catheter of Example 25, wherein the elongated tube transitions from under to over the coupling member on the support member.

In an Example 28, a method of manufacturing a catheter, the method comprising providing an elongated outermost support member of a shaft defining a lumen, the support member having an outer side; and attaching an elongated tube extending longitudinally along the elongated outermost support member; wherein the attaching includes winding a coupling member around the elongated tube and outer side of the support member, the coupling member coupling the elongated tube to the support member.

In an Example 29, the method of Example 28, a further including interfacing the elongated tube with the elongated outermost support member only along the outer side.

In an Example 30, the method of Example 28, and further comprising disposing a mandrel within the elongated tube prior to winding the coupling member.

In an Example 31, the method of Example 28 wherein the elongated tube includes a tube distal end, wherein the tube distal end is closed prior to a cover being applied.

In an Example 32, the method of Example 31, wherein the tube distal end is closed via the coupling member winding around tube distal end and the outer side of the support member.

In an Example 33, the method of Example 32, wherein the cover is applied via a reflow process.

In an Example 34, a system for tracking a catheter during an electrophysiology procedure on a patient, the system comprising a patch electrode mechanically couplable to the patient; a catheter disposable within the patient, the catheter comprising; an elongated shaft defining a lumen and having a distal portion, the elongated shaft having an outermost elongated support member coaxial with the lumen, the support member having an outer side; a tracking electrode disposed on the distal portion of the elongated shaft, the tracking electrode configured to electrically couple with the patch electrode and generate an electrical signal; an elongated tube extending longitudinally along the elongated shaft, the elongated tube interfacing with the support member along the outer side; a lead conductor disposed within the tube and electrically coupled to the electrode, the lead conductor configured to deliver the electrical signal; a coupling member disposed about the tube and outer side of the support member, the coupling member coupling the tube to the support member; and a controller operably coupled to the patch electrode and the tracking electrode, the controller configured to receive the electrical signal and determine a position of the catheter with respect to the patient.

In an Example 35, the system of Example 34, wherein the controller if further configured to generate an electroanatomical map of the heart of the patient. While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2 is a schematic diagram illustrating an example catheter that can be used with the example electrophysiology system. of FIG. 1.

FIG. 3A is a schematic diagram illustrating from a side view a feature of an embodiment of the catheter of FIG. 2.

FIG. 3B is a schematic diagram illustrating from a perspective view the feature of the embodiment of the catheter of FIG. 3A.

FIG. 4 is a schematic diagram illustrating from a side view a feature of another embodiment of the catheter of FIG. 2.

FIG. 5 is a schematic diagram illustrating from a side view a feature of another embodiment of the catheter of FIG. 2.

FIG. 6 is a block diagram illustrating an example method of manufacturing the medical imaging device of FIG. 2.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

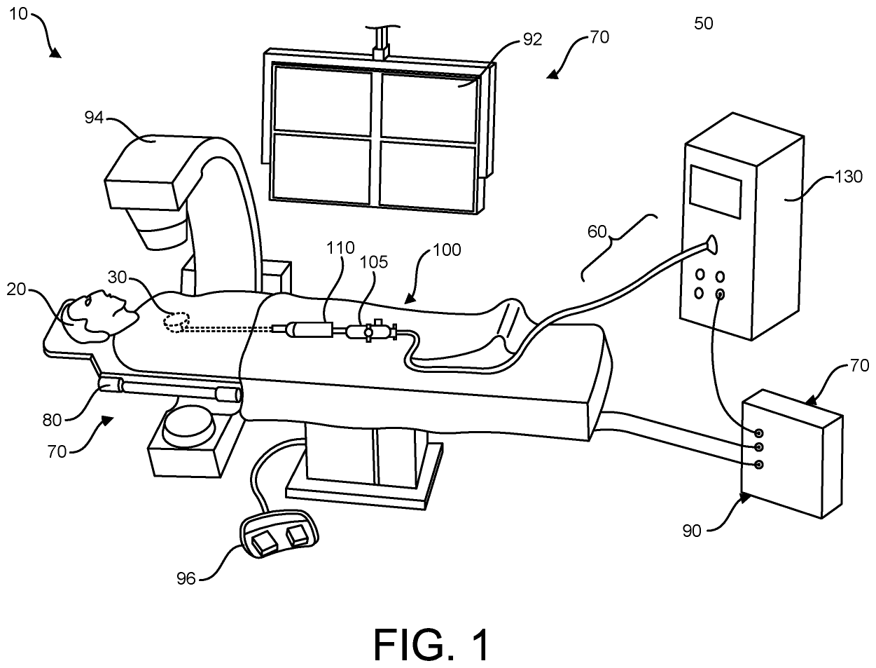

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which in the example includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheters. For instance, a catheter defines a longitudinal axis that passes through a centroid of a cross section of the catheter, such as the centroid of a cross section of a shaft of catheter 105 or a centroid of a cross section of a main lumen of a sheath 110. In the example, the catheter 105 is disposed within the sheath 110. The catheters 105, 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative embodiment, the EAM system 70 can include the RHYTHMIA™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated embodiment, the device tracking is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute tracking electrodes on the tracked catheters that generate outputs received and processed by the mapping and navigation controller 90 to track the location of the various location sensing electrodes within the localization volume. For instance, an impedance tracking methodology can employ the use of a patch electrode (not show) attached to the patient's body, a current or impedance based can be determined between the tracking electrode on the catheter and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters include tracking electrodes disposed on the deflectable portions of the catheter shafts. Multiple tracking electrodes can be employed on the deflectable portions of the catheters for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the catheter such as a conductive braided member along the length of the shaft. Additionally, precise placement of the tracking electrode on the shaft is desired. For instance, the tracking electrode can be radiopaque, and clinicians can use the tracking electrode to visualize placement of the catheter while using fluoroscopy. Also, electrode location with respect to the catheter elements and interelectrode spacing are programmed parameters in several tracking and mapping software programs, and three-dimensional reconstruction and modeling is performed with electrode spacing as a constraint in the modeling curve. Thus, the design and implementation of catheters using tracking electrodes can benefit from ready access to lumens carrying conductor leads.

This disclosure is directed to catheters and methods for assembling catheters that include an insulate tube carrying a conductive lead for a tracking electrode along the side of a support member on a catheter shaft. The tube is coupled to the side of the support member, such as via a coupling member wound over the tube and support member, and the tube, coupling member, and support member can be encapsulated via cover applied with a reflow process. The tubes carrying the conductive electrodes can be readily positioned proximate the tracking electrodes and reduce the likelihood of the conductive lead coming into contact with the conductive support member during manufacturing. This design facilitates easier manufacturing because the tubes are placed outside of the support member (often made from braided stainless steel fibers). Moreover, the tubes are easier to access since they are covered by only a layer of polymer and not positioned under a metallic braided member.

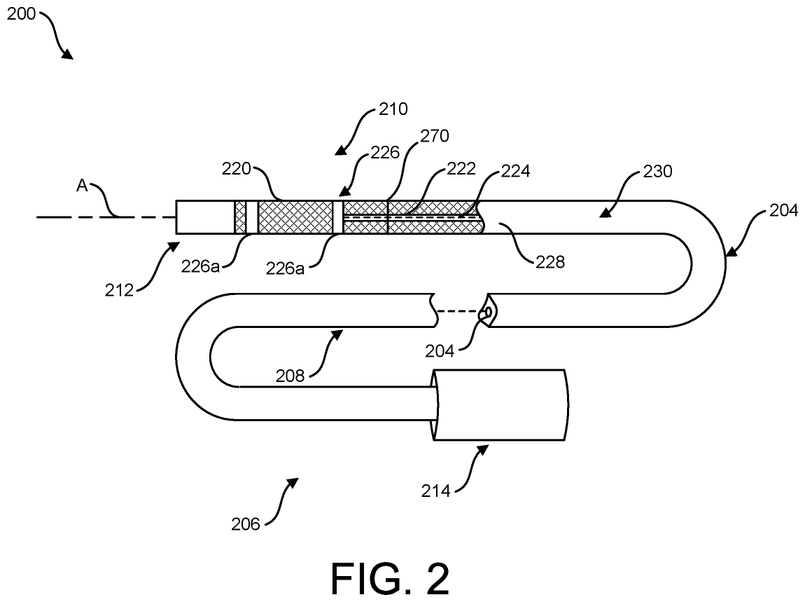

FIG. 2 illustrates an embodiment of a catheter 200 that can be used in the example clinical setting 10 in catheter assembly 100. For instance, catheter 200 can be further configured as a guide catheter, dilator, ablation catheter or other flexible, deflectable medical tool that can be tracked via an impedance tracking system such as EAM system 70. The catheter 200 includes an elongated shaft 202, such as an elongated and flexible shaft 202 defining a longitudinal axis A. The shaft 202 also defines a main lumen 204 along the longitudinal axis A and has a proximal portion 206, a longitudinal section 208, and a distal portion 210. The distal portion 210 includes a distal tip section 212. The proximal portion 206 can be coupled to a handle 214 proximal to the shaft 202. An elongated and flexible support member 220 is disposed over some or all of the shaft 202 along the longitudinal axis. In various embodiments, the support member 220 may be a flexible braid (e.g., stainless steel or a high-strength polymer) or a hypotube (e.g., a laser cut hypotube).

The shaft 202 includes a plurality of components disposed along the distal portion 210. An elongated tube 222 extends longitudinally along the shaft 202 and carries a lead conductor 224 that is coupled to a tracking electrode 226 disposed on the distal portion 208 of the shaft 202. In the illustrated embodiment, the distal portion 210 of the shaft 202 can include a plurality of tracking electrodes 226a . . . 226n, and each tracking electrode 226a . . . 226n can be coupled to a respective lead conductor carried in a corresponding tube extending longitudinally along the shaft 202 and terminated at the proximal portion 206. The tracking electrodes 226 are illustrated as ring electrodes in the shaft. In the example, the tracking electrodes 226a . . . 226n are configured for use with an impedance-based tracking system to detect the position of the catheter. In some embodiments, the tracking electrodes 226 are also radiopaque. A coupling member 270, illustrated schematically, is positioned about the elongated tube 222 and the outer side of the support member 220 to couple the elongated tube 222 to the support member 220. In various embodiments, the coupling member 270 is wound around the outside of the elongated tube 222 and the support member 220.

The proximal portion 206 or handle 212 can include an electrical connection that can be coupled to an impedance-based tracking system, such as EAM system 70. In one example, the electrical connection is available under the trade designation LEMO. The shaft 202 further includes a cover member 228 disposed over the braided member 220 and tube 222 to form an outer surface 230 of the shaft 202.

The catheter 200 can include additional components for a selected implementation. In some embodiments, such as in the case of the catheter 200 configured as a guide sheath or dilator, the proximal portion 206 or handle 214 can include a hemostatic valve that can be coupled to a source of irrigation fluid and a port to receive catheters within the main lumen 204. In some embodiments, the distal tip section 212 can be configured as a dilator tip. In some embodiments, the shaft 202 can include a liner layer (not shown) underneath the support member 220 and coaxial with the main lumen 204. In other embodiments, the liner layer is the support member 220, and the shaft 202 does not include a braided member or other member between the liner layer and the elongated tube 222. The liner layer can define an inner wall of the main lumen 204. In examples such as in which the catheter 200 is a guide sheath or dilator, the liner layer can be a thin wall constructed of polytetrafluoroethylene (PTFE). In some examples, such as in the case of the catheter 200 configured as an ablation catheter, the proximal portion 206 or handle can be coupled to a source of ablation energy, such as an electrical signal from console 130 or a cryogenic fluid. For instance, the main lumen 204 can be configured to carry other electrical leads, such as leads to the ablation electrodes or other sensors, steering wires, or a conduit of irrigation fluid along the shaft 202 to the distal portion 210. In some examples, the distal tip section 212 can be configured to include an ablation electrode assembly or other sensors.

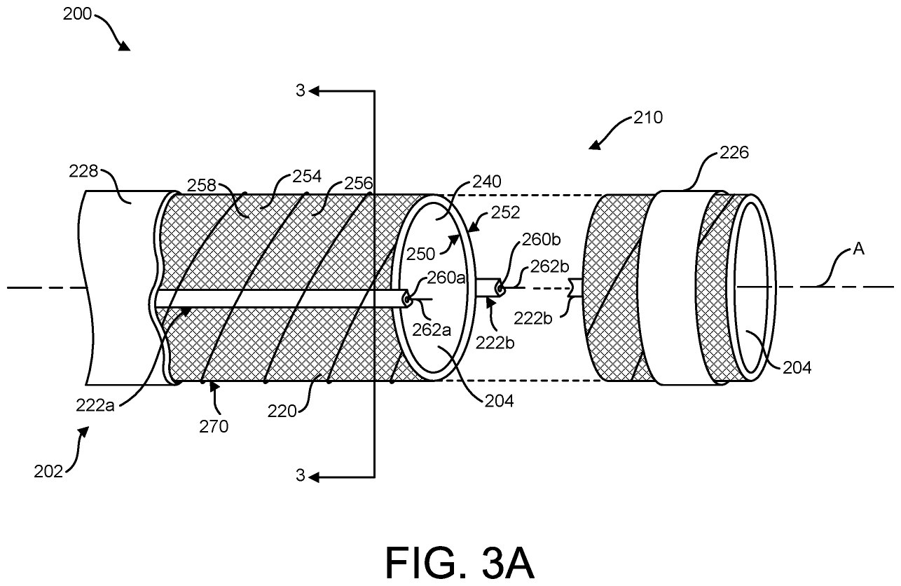

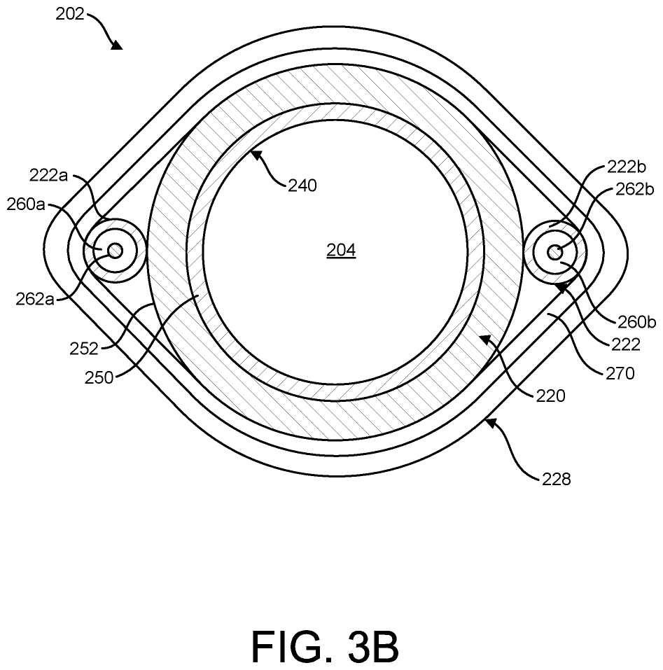

FIG. 3A illustrates features of an embodiment of the catheter 200, and FIG. 3B includes a cross section view of the embodiment of the catheter 200, such as the distal portion 210, taken along line 3-3 of FIG. 3A. In particular, the catheter 200 includes the elongated and flexible shaft 202. The shaft 202 includes the elongated and flexible support member 220 and cover member 228, which are coaxial with the main lumen 204. In some embodiments, the shaft 202 includes liner layer 240 also coaxial with the main lumen 204 and defining an inner wall of the main lumen 204. The support member 220 can extend longitudinally along the shaft 202 and coaxially with the main lumen 204 and disposed on the liner layer 240, for example, such as disposed directly on the liner layer 240. The support member 220 includes an inner side or surface 250 disposed toward the main lumen 204, such as on liner layer 240, and an outer side or surface 252 disposed opposite inner side 250 and the main lumen 204. In some embodiments, the shaft 202 can include a plurality of concentric or coaxially support members, such an innermost support member and an outermost support member 220. In the example, a single support member 220 is also an outermost support member 220.

The support member 220 is illustrated in the embodiment as a braided member 220, which can provide characteristics to the catheter 200 such as reduced kinking, wrinkling, or buckling of the shaft 202 and can provide enhanced balance for pushability, deflectability, and torque transmission such as during rotation about the longitudinal axis A. In another example, the support member 220 can include a coiled shaft member, a laser cut hypotube, the liner layer, or other elongated shaft material. In the illustration, the braided member as the support member 220 is constructed from a woven fabric 254 or layer of braided strands or fibers 256 that form interstitial spaces 258 between the fibers 256. The braided member as the support member 220 can further be characterized by the warp and weft and bias of the fibers 256 as well as picks per inch. For instance, in some embodiments, the picks per inch can remain generally uniform along the entire longitudinal length of the braided member as the support member 220. In some embodiments, the picks per inch can vary along portions of the longitudinal length of the braided member 220. The braided member as the support member 220 can be constructed from fibers 256 that include stainless steel fibers, such as conductive fibers, or high strength polymer fibers, or as layers of different materials.

An elongated tube 222 is coupled to the outermost support member 220 along the outer side 252. For example, the elongated tube 222 extends longitudinally and generally straight along the outer side 252 of the outermost support member 220. The elongated tube 222 contacts with the outer side 252 of the support member 220 such as is abutted against the material of the support member 220 or is glued to the support member 220. The elongated tube 222 is configured from an insulate material, such as a polymer, and defines a longitudinally extending lead lumen 260 along the length of the tube 222. The tube 222 is configured to carry a lead conductor, such as lead wire 262, within the lead lumen 260. The lead wire 262 is configured to be electrically coupled to a tracking electrode 226 and carry an appropriate electrical signal from the electrical connector at the proximal portion 204 to the tracking electrode 226. In one embodiment, the elongate tube 222 is of a substantially smaller diameter than the shaft 202, and the main lumen 204 has a substantially larger diameter than the diameter of the lead lumen 260. In an embodiment of a catheter 200 including multiple tracking electrodes, an elongated tube carrying a lead wire is included for each tracking electrode. For instance, a catheter can include two tracking electrodes and includes a first elongated tube 222a carrying a first lead wire 262a within first lead lumen 260a and a second elongated tube 222b carrying a second lead wire 262b within first lead lumen 260b. Multiple elongated tubes 222 can be radially spaced-apart on the outer side 252 of the braided member 220. In the illustrated embodiment, the elongated tubes 222a, 222b are radially spaced 180 degrees apart. The lead wires 262 are disposed within the elongated tubes 222 either as bare wire or with an insulation covering. Each lead lumen 260 terminates proximate or at the corresponding tracking electrode.

The elongated tube 222 contacts the outermost support member 220 along the outer side 252. In one embodiment, the elongated tube 222 contacts with the outer side 252 of the outermost support member 220 and does not traverse between an innermost and outermost support member or into the lumen 204 along the entire length of the shaft 202. In another embodiment, the elongated tube 222 contacts with the outer side 252 of the outermost support member 220 in the distal portion 210, but otherwise traverses underneath the outermost support member 220 in the proximal portion 206. For example, the tube 222 can extend through an interstitial space 258 between the fibers 256 and travel into the lumen 240 or between inner and outer braided members.

A coupling member 270 is positioned about the elongated tube 222 and the outer side 252 of the support member 220 to couple the elongated tube 222 to the support member 220. In various embodiments, the coupling member 270 is wound around the outside of the elongated tube 222 and the support member 220 to couple the elongated tube 222 to the support member 220. In other embodiments, the coupling member 270 is formed into a braid and is place about the outside of the elongated tube 222 and the support member 220. In one example, the coupling member 270 couples the elongated tube 222 to the outer side of the support member 220 during manufacturing of the shaft 202 until at least a cover member 228 is disposed on the support member 220. For instance, the coupling member 270 can be wound, coiled or braided around or coiled around the support member 220.

The coupling member 270 (e.g., a thread, filament or braid) can be constructed from a suitable material, and in one embodiment is from a material that is of a tensile strength to not break while the tube 222 is being coupled to the support member 220 and when subjected to additional manufacturing processes. For example, the material can include a high melting temperature that can withstand a reflow process to apply the cover member 228. In one embodiment, the coupling member 270 can include a nylon type 6,6 having a monofilament construction and a size USP 6-0, which has a demonstrated tensile strength and resists melting during reflow temperatures. Additionally, a clear thread can remain hidden after manufacturing of the shaft 202 and does not interfere with additional manufacturing processes such as drilling holes into the shaft such as into the support member 220. In additional embodiments, the thread can be selected to be radiolucent, nonconductive, or both. In one embodiment, a single pass of the coil along the length of the elongated tube 222 with a pitch of four millimeters was sufficient to secure the tube 222 to the support member 220.

The cover 228, or outer layer, is disposed on the support member 220, elongated tube 222, and coupling member 270. In some embodiments, the cover 228 can be formed as a coating of reflowable plastic or thermoplastic material that extends over the support member 220, elongated tube 222, and coupling member 270 and seals underlying components of the shaft 202. For instance, the coating can seep by reflowing over braided material of the support member 220 such as over the fibers 256 and coupling member 270 and into the interstitial spaces 258 of the braided material of the support member 220 and onto the tubes 222. In one example, the cover 228 is a polyether block amide and, in some examples, is available under the trade designations PEBAX from Arkema, S.A., and VESTAMID E from Evonik Industries, AG.

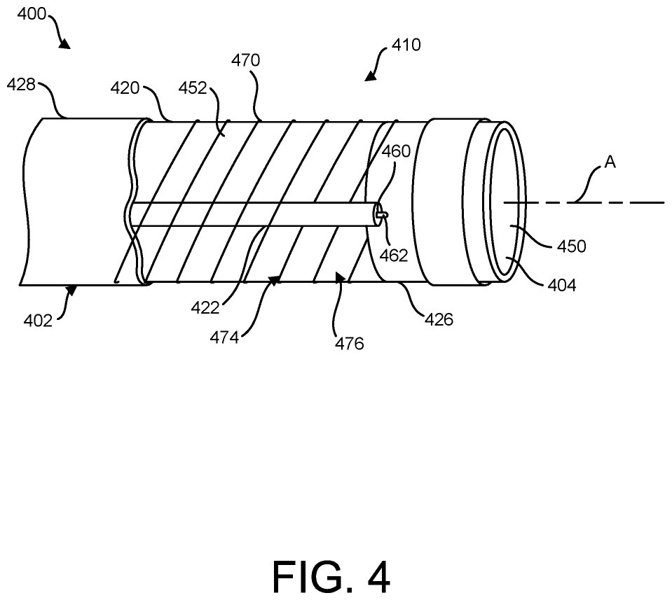

FIG. 4 illustrates features of another embodiment of the catheter 200, or catheter 400 having a distal portion 410. In particular, the catheter 400 includes an elongated and flexible shaft 402. The shaft 402 includes the elongated and flexible outermost support member 420 and cover member 428, which are coaxial with the main lumen 404. The support member 420 includes an inner side or surface 450 disposed toward the main lumen 404, and an outer side or surface 452 disposed opposite inner side 450 and the main lumen 404. Embodiments of the support member include a braided member, a coiled member, a laser cut tube, and a liner layer. In some embodiments, the shaft 402 can include a plurality of concentric or coaxially support members, such an innermost support member and an outermost support member 420. A single support member 420 is also an outermost support member 420.

An elongated tube 422 extending longitudinally along the elongated shaft 402 is coupled to the outermost support member 420 along the outer side 452. For example, the elongated tube 422 extends longitudinally and generally straight along the outer side 452 of the outermost support member 420. The elongated tube 422 contacts or interfaces with the outer side 452 of the support member 420 such as is abutted against the material of the support member 420 or is glued to the support member 420. The elongated tube 422 is configured from an insulate material, such as a polymer, and defines a longitudinally extending lead lumen 460 along the length of the tube 422. The tube 422 is configured to carry a lead conductor, such as lead wire 462, within the lead lumen 460. The lead wire 462 is electrically coupled to a tracking electrode 426 and is configured to carry an appropriate electrical signal from the electrical connector at the proximal portion to the tracking electrode 426. The lead wire 462 is disposed within the elongated tube 422 either as bare wire or with an insulation covering. The lead lumen 460 terminates proximate or at the corresponding tracking electrode 426.

A coupling member 470 is positioned about the elongated tube 422 and the outer side 452 of the support member 420 to couple the elongated tube 422 to the support member 420. In various embodiments, the coupling member 470 is wound around the outside of the elongated tube 422 and the support member 420 to couple the elongated tube 422 to the support member 420. In the illustrated embodiment, the coupling member 470 is wound around the elongated tube 422 and support member 420 in a single coil along the length of the shaft 402 or at least a section of the distal portion 410 as illustrated. The coupling member 470 (e.g., a thread, filament or braid) can be constructed from a suitable material, and in one embodiment is from a material that is of a tensile strength to not break while the tube 422 is being coupled to the support member 420 and when subjected to additional manufacturing processes. In one embodiment, the coupling member 570 can include a nylon type 6,6 having a monofilament construction and a size USP 6-0, which has a demonstrated tensile strength and resists melting during reflow temperatures.

The wound coupling member 470 can provide additional support to the shaft 402, particularly if the shaft 402 does not include a braided fabric support member 420 and the support member 420 is a liner layer. In this embodiment, the coupling member forms a reinforcing sheath and the coil is wound with a suitable pitch to provide stability. The wound coupling member 470 provides enhanced balance for pushability, deflectability, and torque transmission such as during rotation about the longitudinal axis A in lieu of the braided fabric support member 420. In one embodiment, the wound coupling member 470 can extend distal to the distal most tracking electrode 426 to provide support. In the illustrated example, the coupling member 470 is wound over the elongated tube 422 and support member in a first section 474 of the support member 420 on the distal portion 410 and wound under the elongated tube 422 and over the support member 420 in a second section 476 of the support member 420 distal to the first section 474. In the illustrated embodiment, the elongated tube 422 transitions from under the coupling member 470 at the first section 474 to over the coupling member 470 at the second section 476 on the distal portion 410.

The cover member 428, or outer layer, is disposed on the support member 420, elongated tube 422, and coupling member 470. In some embodiments, the cover 428 can be formed as a coating of reflowable plastic or thermoplastic material that extends over the support member 420, elongated tube 422, and coupling member 470 and seals underlying components of the shaft 402.

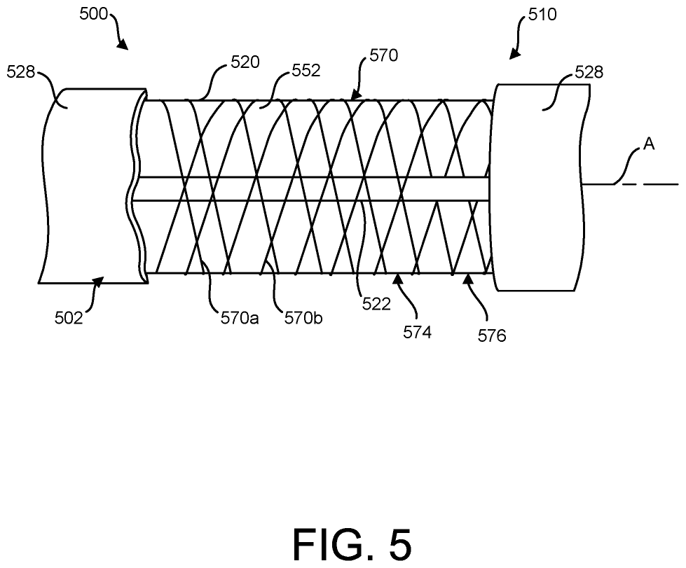

FIG. 5 illustrates features of another embodiment of the catheter 200, or catheter 500 having a distal portion 510. In particular, the catheter 500 includes an elongated and flexible shaft 502. The shaft 502 includes the elongated and flexible outermost support member 520 and cover member 528, which are coaxial with the main lumen. The support member 520 includes an outer side or surface 552 disposed opposite an inner side and the main lumen. An elongated tube 522 extending longitudinally along the elongated shaft 502 is coupled to the outermost support member 520 along the outer side 552. The tube 522 is configured to carry a lead conductor, such as lead wire, within the lead lumen, and the lead wire is electrically coupled to a tracking electrode (not shown).

A coupling member 570 is positioned about the elongated tube 522 and the outer side 552 of the support member 450 to couple the elongated tube 522 to the support member 520. In the embodiment, the coupling member 570 includes a plurality of threads, filaments, or braids each wound around the outside of the elongated tube 522 and the support member 520 to couple the elongated tube 522 to the support member 520. In the illustrated embodiment, the coupling member 570 is wound around the elongated tube 422 and support member 420 as a first coil 570a having a first pitch and in a first direction and as a second coil 570b having a second pitch and in a second direction opposite the first direction. For instance, the first coil 570a can be wound in a clockwise direction along a length of the support member 520 and the second 570b is wound in a counterclockwise direction along the length of the support member 520. The coupling member 570 (e.g., a thread, filament or braid) can be constructed from a suitable material, and in one embodiment is from a material that is of a tensile strength to not break while the tube 522 is being coupled to the support member 520 and when subjected to additional manufacturing processes. The coils 570a, 570b can be of dissimilar materials or the same material. In one embodiment, the coupling member 570 can include a nylon type 6,6 having a monofilament construction and a size USP 6-0, which has a demonstrated tensile strength and resists melting during reflow temperatures. The wound coupling member 570 with a plurality of coils can provide additional support to the shaft 502 over a single coil, particularly if the shaft 502 does not include a braided fabric support member 520 and the support member 520 is a liner layer. In this embodiment, the coupling member forms a reinforcing sheath and the coils are wound with suitable pitches to provide stability. The wound coupling member 570 provides enhanced balance for pushability, deflectability, and torque transmission such as during rotation about the longitudinal axis A in lieu of the braided fabric support member 520. In the illustrated example, the coupling member 570 is wound over the elongated tube 522 and support member in a first section 574 of the support member 520 on the distal portion 510 and wound under the elongated tube 522 and over the support member 520 in a second section 576 of the support member 520 distal to the first section 574. In the illustrated embodiment, the elongated tube 522 transitions from under the coupling member 570 at the first section 574 to over the coupling member 570 at the second section 576 on the distal portion 510.

The cover member 528, or outer layer, is disposed on the support member 520, elongated tube 522, and plurality of coils of the coupling member 570. In some embodiments, the cover 528 can be formed as a coating of reflowable plastic or thermoplastic material that extends over the support member 520, elongated tube 522, and plurality of coils of the coupling member 570 and seals underlying components of the shaft 502.

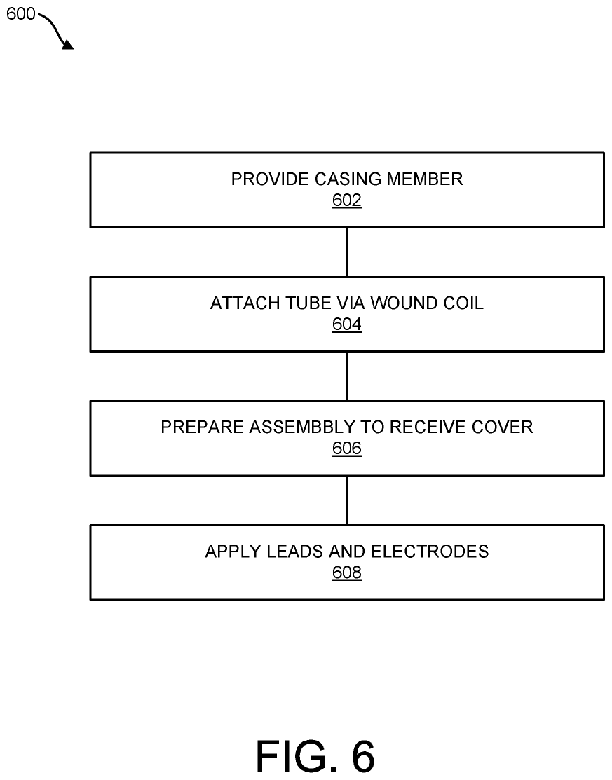

FIG. 6 illustrates an example method 600 of manufacturing a catheter. In one example, method 600 includes a method of constructing a catheter shaft having longitudinal support member with an outer side and defining a lumen, the outer side of the support member coupled to a tube defining a lead lumen including a conductor lead configured to be attached to a tracking electrode on the catheter shaft. In one embodiment, an elongated support member, such as support member 220 (e.g., a casing member) is provided at 602. The support member 220 can include a braided member. An elongated tube extending longitudinally is coupled (e.g., attached) along the elongated support member, for example, by winding a thread around the tube and outer side of the support member to create an assembly at 604. A tool can be applied to the tube and support member to align the tube longitudinally along the tube during winding of the thread around the tube and the outer surface. A mandrel can be disposed in the tube while the thread is being wound on the tube and the outer surface to wind the thread tightly but maintain the lead lumen in the tube. In one embodiment, the mandrel can be removed after the winding is complete.

The tube and support member assembly are prepared to receive a cover at 606. In this example, a distal end of the tube is sealed to protect the lead lumen while the cover is being applied. For example, the distal end of the tube can extend past the mandrel, and the distal end can be tightly wound against the outer side with the thread to seal the lead lumen of the tube. Other embodiments of sealing the distal end of the tube include applying a glue in the lead lumen at the distal end. The distal end of the tube is sealed to reduce the likelihood of the cover filling the lead lumen. Additionally, the ends of the tube can be reinforced to remain under tension against the elongate support member in preparation for the cover. In one example, the ends of the tube are secured to the support member via an attachment member including tape, such as silicone tape, knots, melted extrusions, or a heat shrink tubing applied over the ends and heated. An example of the heat shrink tubing can include polyethylene terephthalate (PET) medical heat shrink tubing. The cover can be applied via reflow. The attachment member can be removed after the cover is applied. A lead conductor is put into a lead lumen of the tube, and the lead conductor is electrically coupled to a tracking electrode disposed on the shaft at 608.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter, comprising:
an elongated shaft defining a lumen and having a distal portion, the elongated shaft having an outermost elongated support member coaxial with the lumen, the support member having an outer side;
an electrode disposed on the distal portion of the elongated shaft;
an elongated tube extending longitudinally along the elongated shaft, the elongated tube coupled to the support member along the outer side;
a lead conductor disposed within the tube and electrically coupled to the electrode; and
a coupling member disposed about the tube and outer side of the support member, the coupling member coupling the tube to the support member.

2. The catheter of claim 1, wherein the support member includes a braided material forming a braided member.

3. The catheter of claim 2, wherein the braided member includes conductive fibers.

4. The catheter of claim 3, wherein the coupling member is a thread.

5. The catheter of claim 1, wherein the elongate tube traverses into the lumen through the braided member on a proximal side of the shaft.

6. The catheter of claim 1, wherein the shaft includes a cover disposed on the coupling member, tube, and support member.

7. The catheter of claim 6, wherein the cover includes a polyether block amide and the coupling member includes a nylon thread.

8. The catheter of claim 1, wherein the elongate tube includes a plurality of elongate tubes radially spaced-apart on the outer side.

9. The catheter of claim 1, wherein the support member is a liner layer.

10. The catheter of claim 1, wherein the coupling member includes one or more coils wound around the elongated tube and support member.

11. The catheter of claim 10, wherein the coupling member includes a first coil and a second coil, the first coil wound in a first direction along a length of the support member and the coil wound in a second direction along the length of the support member, the second direction opposite the first direction.

12. The catheter of claim 10, wherein the elongated tube transitions from under to over the coupling member on the support member.

13. A method of manufacturing a catheter, the method comprising:
providing an elongated outermost support member of a shaft defining a lumen, the support member having an outer side; and
attaching an elongated tube extending longitudinally along the elongated outermost support member;
wherein the attaching includes winding a coupling member around the elongated tube and outer side of the support member, the thread coupling the elongated tube to the support member.

14. The method of claim 13, a further including interfacing the elongated tube with the elongated outermost support member only along the outer side.

15. The method of claim 13, and further comprising disposing a mandrel within the elongated tube prior to winding the coupling member.

16. The method of claim 13, wherein the elongated tube includes a tube distal end, wherein the tube distal end is closed prior to a cover being applied.

17. The method of claim 16, wherein the tube distal end is closed via the coupling member winding around tube distal end and the outer side of the support member.

18. The method of claim 17, wherein the cover is applied via a reflow process.

19. A system for tracking a catheter during an electrophysiology procedure on a patient, the system comprising:
a patch electrode mechanically couplable to the patient;
a catheter disposable within the patient, the catheter comprising:
an elongated shaft defining a lumen and having a distal portion, the elongated shaft having an outermost elongated support member coaxial with the lumen, the support member having an outer side;
a tracking electrode disposed on the distal portion of the elongated shaft, the tracking electrode configured to electrically couple with the patch electrode and generate an electrical signal;
an elongated tube extending longitudinally along the elongated shaft, the elongated tube interfacing with the support member along the outer side;
a lead conductor disposed within the tube and electrically coupled to the electrode, the lead conductor configured to deliver the electrical signal; and
a coupling member disposed about the tube and outer side of the support member, the coupling member coupling the tube to the support member; and
a controller operably coupled to the patch electrode and the tracking electrode, the controller configured to receive the electrical signal and determine a position of the catheter with respect to the patient.

20. The system of claim 19, wherein the controller if further configured to generate an electroanatomical map of the heart of the patient.
